(19) United States
(12) Patent Application Publication
(10) Pub. No.: US 2014/0058359 A1
(43) Pub. Date: Feb. 27, 2014

(54) INTRODUCER SHEATH FOR PERFUSION REGULATION SYSTEM

(71) Applicant: COOK MEDICAL TECHNOLOGIES LLC, Bloomington, IN (US)

(72) Inventors: Vanessa Weisman, Bloomington, IN (US); Karol Lagodzki, Bloomington, IN (US)

(73) Assignee: COOK MEDICAL TECHNOLOGIES LLC, Bloomington, IN (US)

(21) Appl. No.: 13/967,791

(22) Filed: Aug. 15, 2013

Related U.S. Application Data
(60) Provisional application No. 61/692,012, filed on Aug. 22, 2012.

(51) Int. Cl. A61M 25/10

(52) U.S. Cl. CPC A61M 25/10
USPC 604/510; 604/103.05

(57) ABSTRACT

A reperfusion system and method of perfusing a blood vessel using the reperfusion system are provided. A sheath has a sheath wall and a lumen that each extend to a distal end to define a distal opening. A distal portion of the sheath wall has at least one wall opening. A balloon catheter has a lumen that extends to the distal end to define a distal opening. The balloon catheter has an inflatable balloon attached to a distal portion. A conduit has a first end in fluid communication with the lumen of the sheath and a second end in fluid communication with the lumen of the balloon catheter. The reperfusion system is configured to allow blood to enter each of the distal opening and the at least one wall opening of the sheath, flow through the sheath, the conduit, and the balloon catheter, and exit through the distal opening of the balloon catheter. The sheath is introduced into body tissue. The balloon catheter is advanced through the sheath to the blood vessel. The balloon is inflated. Blood is allowed to enter each of the distal opening and the at least one wall opening of the sheath, flow through the sheath, the conduit, and the balloon catheter, and exit through the distal opening of the balloon catheter.

### CROSS-REFERENCE TO RELATED APPLICATIONS

This application claims the benefit of U.S. Provisional Application No. 61/692,012, filed on Aug. 22, 2012, entitled “INTRODUCER SHEATH FOR PERFUSION REGULATION SYSTEM,” the entire contents of which are incorporated herein by reference.

### FIELD OF THE INVENTION

The present disclosure generally relates to medical devices. More specifically, the present disclosure relates to perfusion regulation systems.

### BACKGROUND

In standard catheter embolization procedures, backflow may cause non-target embolization. This backflow may be caused by inadequate techniques employed by the person performing the treatment. Also, where pressure builds up distally such as with embolization procedures with microparticles and liquid embolic agents (glue/Onyx [Ev3]) backflow may result. This backflow may also result in non-target embolization. In order to prevent backflow, an occlusion balloon catheter may be used in the target vessel. However, inflation of the balloon stops the blood flow to the organs and or tissues of the target vessel which introduces additional risks.

### SUMMARY

In overcoming the drawbacks and other limitations of the related art, the present disclosure provides artificial antegrade flow in the target vessel. The flow provides perfusion to the tissue and organs and is used to aid the embolic agent in reaching the target area. The present disclosure simultaneously combines no back flow when the balloon is insufflated, full embolization control, and enhanced safety. Once the artificial antegrade blood vessel flow is established, a micro catheter may be introduced through the occlusion balloon catheter, in a coaxial manner, up to the target blood vessel and embolization using standard techniques may be performed. The flow into the introducer sheath is enhanced by including openings on the introducer sheath, for example at the distal portion of the introducer sheath.

The present disclosure provides a reperfusion system. A sheath has a sheath wall that extends from a proximal portion to a distal portion that has a distal end. The sheath has a lumen that extends to the distal end to define a distal opening of the sheath. The distal portion of the sheath wall having a plurality of wall openings that are one of circumferentially and longitudinally spaced apart. A catheter has a catheter wall that extends from a proximal portion to a distal portion that has a distal end. The catheter has a lumen that extends to the distal end to define a distal opening of the catheter. The catheter is disposed in the lumen of the sheath. The catheter has an expandable member attached to the distal portion. A conduit has a first end in fluid communication with the lumen of the sheath between the sheath wall and the catheter wall, and a second end in fluid communication with the lumen of the catheter. The reperfusion system is configured to allow blood to enter each of the distal opening and the at least one wall opening of the sheath, flow through the sheath, the conduit, and the catheter, and exit through the distal opening of the catheter.

The present disclosure also provides a method of perfusing a blood vessel. The method includes providing the reperfusion system. The method further includes introducing the sheath in body tissue. The method further includes advancing the catheter through the sheath to the blood vessel. The method further includes expanding the expandable member to prevent blood from flowing in the blood vessel. The method further includes allowing the blood to enter each of the distal opening and the at least one wall opening of the sheath, flow through the sheath, the conduit, and the catheter, and exit through the distal opening of the catheter.

Further features and advantages of the present disclosure will become apparent from consideration of the following description and the appended claims when taken in connection with the accompanying drawings. It should be understood that the description and specific examples are intended for purposes of illustration only and are not intended to limit the scope of the present disclosure.

### BRIEF DESCRIPTION OF THE DRAWINGS

FIG. 1 is a side schematic view of a reperfusion system;

FIG. 2 is a cross-sectional view taken along lines 2-2 of FIG. 1;

FIG. 3 is a cross-sectional view taken along lines 3-3 of FIG. 1;

FIG. 4 is a side schematic view of an introducer sheath;

FIG. 5 is a side schematic view of another introducer sheath;

FIG. 6 is a side schematic view of another introducer sheath; and

FIG. 7 is a side schematic view of another introducer sheath.

The drawings described herein are for illustration purposes only and are not intended to limit the scope of the present disclosure in any way.

### DETAILED DESCRIPTION

The present disclosure generally relates to a reperfusion system having an introducer sheath which has openings at its distal portion to optimize blood flow into the introducer sheath and through the reperfusion system. The reperfusion system can be used in all types of embolization procedures including, but not limited to, chemo-embolization, radio-embolization, embolization of high flow blood vessels, situations where there is a high risk of non-target embolization, apparently safe situations in which a non-target embolization might lead to severe complications, and interventional oncology procedures where microparticles (a type of embolic agent) with chemotherapy or with radiation are injected into tumor vessels.

The term “substantially” used herein with reference to a shape includes variations in the recited shape that are equivalent to the shape for an intended purpose or function.

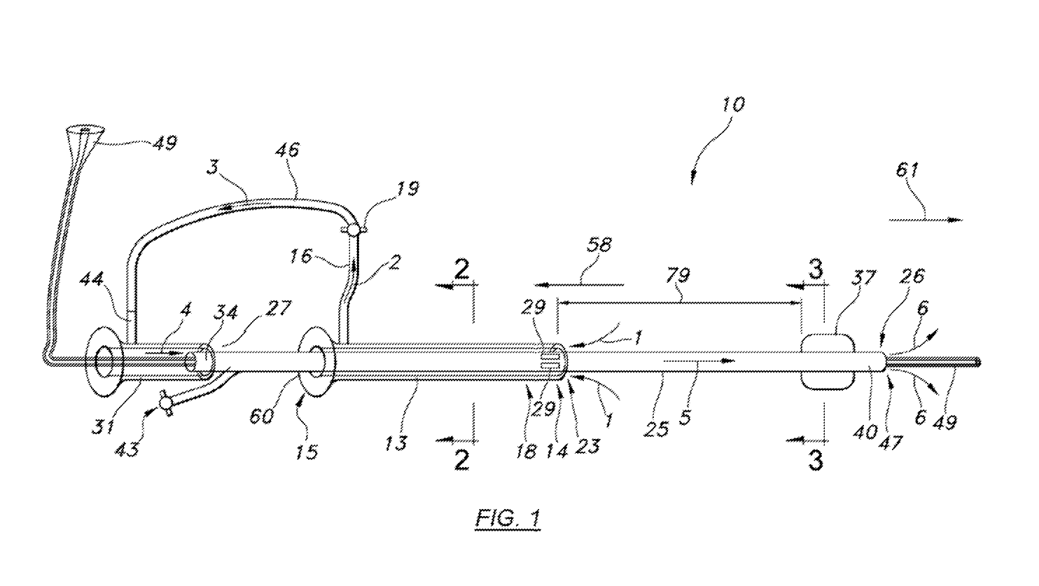

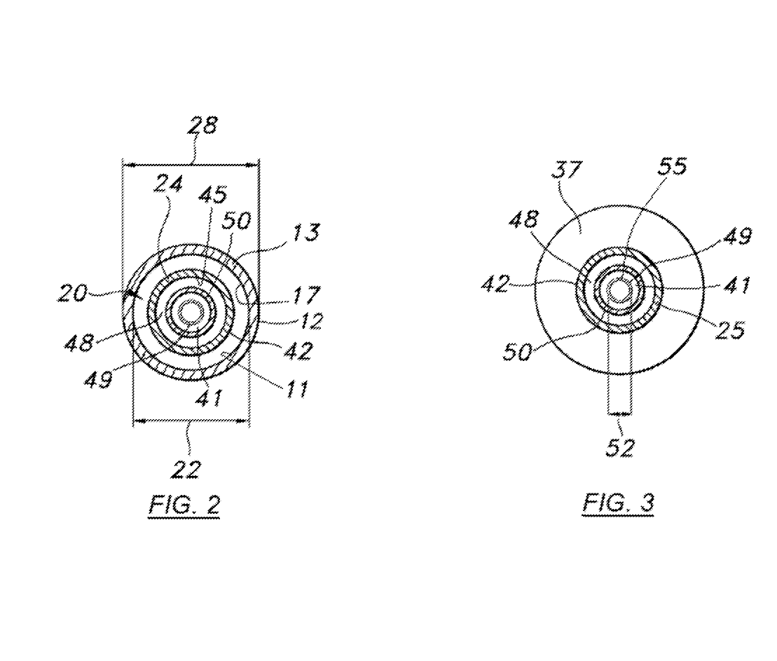

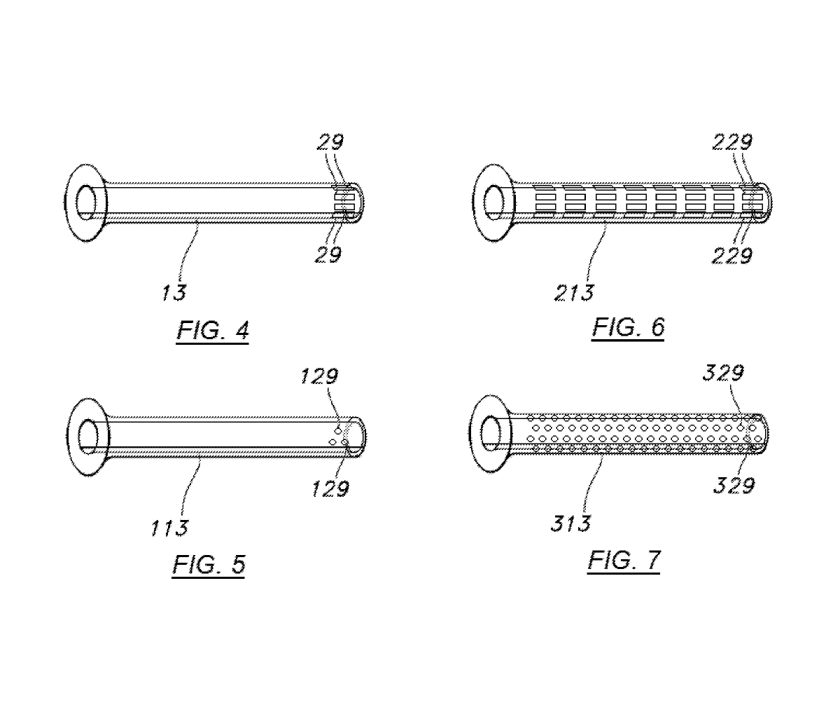

FIGS. 1-3 illustrate a reperfusion catheter system 10 which includes an introducer sheath 13 having a sheath wall 17 that extends from a proximal portion 15 to a distal portion 18 that has a distal end 14. The sheath wall 17 has an inner surface 11 and an outer surface 12. The inner surface 11 of the sheath wall 17 defines a longitudinal lumen 20 that extends to the distal end 14 to define a distal opening 23 of the introducer sheath 13. The distal portion has wall openings 29 (i.e. holes or perforations), which will be described in more detail in FIGS. 4-8.

The introducer sheath 13 may be made of polytetrafluoroethylene (PTFE), radiopaque fluorinated ethylene propylene (FEP), or combinations thereof, for example. The introducer sheath 13 may have any suitable size, for example about 5 French, about 8 French, or between about 5 French and about 8 French. The introducer sheath 13 may have an inner diameter 22 of about 0.047 inches and an outer diameter of about 0.094 inches, or it may have an inner diameter 22 of about 0.113 inches and an outer diameter of about 0.133 inches. Thus, the inner diameter 22 may be between about 0.074 inches and about 0.113 inches, and the outer diameter 28 may be between about 0.094 inches and about 0.133 inches, for example. The introducer sheath 13 may be shorter in length than a typical introducer sheath in order to maximize the artificial antegrade blood flow as described in greater detail below. The introducer sheath 13 has the largest inner diameter 22 possible in order to allow antegrade blood flow around an occlusion catheter 25 (e.g. occlusion balloon catheter) that is inserted through the opening 20 in the introducer sheath 13. The outer diameter 28 of the introducer sheath 13 is as small as possible in order to have a low profile and to minimize puncture site complications. The proximal portion 15 is attached to a lateral check flow 16 which includes a stopcock 19. The lateral check flow 16 is a side port that may be integrally formed with or attached to the introducer sheath 13.

The flexible occlusion balloon catheter 25 has a catheter wall 24 that extends from a proximal portion 27 to a distal portion 40 having a distal end 26. The catheter wall 24 has an inner surface 45 and an outer surface 42. The catheter 25 includes a longitudinal lumen 41 that extends to the distal end 26 to define a distal opening 47 of the balloon catheter 25.

The balloon catheter 25, including the catheter wall 24, may be made of radiopaque vinyl (e.g. polyvinyl chloride), nylon, urethane, or combinations thereof, for example. The balloon catheter 25 may have any suitable size, for example about 5.3 French, or between about 3 French and about 5.3 French. The balloon catheter 25 may have an inner diameter of about 0.038 inches and an outer diameter of about 0.072 inches, or it may have an outer diameter of about 0.039 inches. Thus, the inner diameter may be equal to or less than about 0.038 inches, and the outer diameter may be between about 0.039 inches and about 0.072 inches.

A detachable valve system 31 (for example, a Touhy-Borst adapter) is attached to a hub 34 in the proximal portion 27 of the balloon catheter 25. An expandable member 37 (for example, an inflatable balloon) is attached to the distal portion 40 of the balloon catheter 25. The balloon 37 may be made of polyurethane, nylon, or latex, for example. A side port 43 intersects with the balloon catheter 25 at the proximal portion 27. The inner surface 45 of the catheter wall 24 defines an inflation lumen 48 that extends from the intersection to the inside of the balloon 37. The lumen 41 is coaxially located inside the inflation lumen 48. A wall 50 separates the inflation lumen 41 and the lumen 48. In other embodiments, the inflation lumen 48 may be located coaxially inside the lumen 41. In other embodiments, rather than being coaxial, the lumen 41 and the inflation lumen 48 may be non-coaxially spaced away from each other inside the catheter 25. The balloon 37 can be expanded (e.g. inflated) by providing fluid through the side port 43 and through the inflation lumen 48. The valve system 31 has a side port 44 which is attached to a connecting tube 46 that extends to and is attached to the lateral check flow 16 of the introducer sheath 13. Thus, the side port 44, connecting tube 46, and the lateral check flow 16 form a conduit that allows blood to flow from the introducer sheath 13 (which, for example, may be located in the lumen of the femoral artery) through the connecting tube 46 into the balloon catheter 25 and exiting through the distal opening 47 of the balloon catheter 25 at the target vessel location.

A micro catheter 49 may be inserted through the lumen 41 in the occlusion balloon catheter 25 to allow embolization with microparticles, micro coils or liquid embolic agents (glue or Onyx [Ev3]). The micro catheter 49, including its outer wall 55, may be made of PTFE, metal braids, nylon, PEBAX, or combinations thereof, for example. The micro catheter 49 may have any suitable size, for example about 2.3 French, or equal to or less than about 2.3 French. The outer diameter 52 of the micro catheter 49 may be about 0.03 inches, or equal to or less than about 0.03 inches. The outer diameter 52 (FIG. 3) is preferably minimized. Also, the outer wall 55 (FIG. 3) of the micro catheter 49 is preferably made as thin as possible as long as there is no compromise in push ability and the ability to deliver embolic agents. Alternatively, embolic agents, for example 0.035″ coils, can be delivered through the occlusion balloon catheter 25. The catheter system 10 of the present invention is preferably comprised of polymers that provide the specific features of diameter compatibility, flexibility, torque-ability, and optimized blood flow.

FIGS. 4-7 illustrate introducer sheaths 13, 113, 213, 313, each having different patterns of openings 29, 129, 229, 329 on the sheath wall 17, and each of which can be used with the reperfusion system 10. Each of the openings 29, 129, 229, 329 extend from the inner surface 11 to the outer surface 12 of the introducer sheaths 13, 113, 213, 313. The patterns and shapes of the openings may be designed to optimize blood flow into the introducer sheath 13, 113, 213, 313 and through the reperfusion system 10. The FIG. 4 shows rectangular openings 29 which are circumferentially spaced apart around the entire circumference of the introducer sheath 13. FIG. 5 shows circular openings 129, two of which are longitudinally spaced apart on the introducer sheath 113. The circular openings 129 may also be circumferentially spaced apart the entire circumference of the introducer sheath 113. Additionally, as shown in FIGS. 4 and 5, the openings 29, 129 are formed only on the distal portion 18 of the introducer sheath 13, 113. In variations of the patterns of FIGS. 4 and 5, the openings 29, 129 may be spaced around only part of the circumference rather than the entire circumference. In variations of the introducer sheaths 13, 113 of FIGS. 4 and 5, the introducer sheaths 213, 313 show the openings 229, 329 spaced longitudinally along the entire length of the introducer sheaths 213, 313. Additionally, the openings 229, 329 may be circumferentially spaced apart around a part of or the entire circumference of the introducer sheaths 213, 313. In some embodiments, the openings 29, 129, 229, 329 may be located only on the proximal portion 13 of the sheaths 13, 113, 213, 313.

The shape of each opening may be a polygon or substantially a polygon, triangle or substantially a triangle, quadrilateral or substantially a quadrilateral, square or substantially a square, rectangle or substantially a rectangle, pentagon or substantially a pentagon, a hexagon or substantially a hexagon, an octagon or substantially an octagon, a circle or substantially a circle, an oval or substantially an oval, or an irregular shape. In some examples, a particular introducer sheath may openings of more than one shape, where the possible combinations may include any of the above shapes.

In operation the system is used as follows. Once vascular access has been obtained (either arterial/venous), the introducer sheath 13 is introduced into body tissue, for example a blood vessel such as the femoral artery. In the lumen of the blood vessel (e.g. the femoral artery) where the introducer sheath 13 is introduced, the normal blood flow is in the direction of arrow 58, for example if arterial entry of the introducer sheath 13 is done in the superior direction. Using standard catheterization techniques, the expandable member 25 (e.g. occlusion balloon catheter 25) is advanced selectively up to a target vessel. At the lumen of the target vessel, the normal blood flow is in the direction of arrow 61. The blood flow may be in the direction of arrow 61 in both the vessel where the introducer sheath 13 is introduced, and in the target vessel, for example if arterial entry of the introducer sheath 13 is done in the inferior direction. In such examples, these entry vessel and target vessel may also, for example, be the same vessel.

The distance between the position where vascular access is obtained (e.g., the femoral artery) and the target vessel varies depending on the location of the target vessel. Accordingly, the length 79 of the balloon catheter 25 between the distal end 14 of the introducer sheath 13 and the balloon 37 will vary depending on how far the balloon catheter 25 is extended into the vasculature. Once the balloon catheter 25 is deployed to the target vessel, the lateral check flow 16 of the introducer sheath 13 may be connected into the valve system 31 (for example, a Tuohy-Borst adaptor) by means of the connecting tube 46. An adapter such as, for example, a Luer-lock attaches the valve system 31 to the hub 34 of the occlusion balloon catheter 25. At this point, to stop the blood flow in the target vessel, the expandable member 37 (e.g. balloon 37) is expanded (e.g. insufflated, and thereby inflated) by providing fluid through the side port 43 and the inflation lumen 48. The stopcock 19 can be switched from a closed position to an open position to allow blood from the common femoral artery to flow through the lateral check flow 16 and the valve system and through the reperfusion system 10 into the target vessel.

The above-identified technique provides an artificial/diverted antegrade flow towards the target vessel that is important to aid the embolic agent in reaching the target area distally, such as for treating a tumor microvasculature. Without having back blood flow in the target vessel when the balloon 37 of the occlusion balloon catheter 25 is insufflated, there is a remarkable enhancement in procedure safety (antegrade flow, no back flow).

The flow path for the blood is indicated by arrows numbered 1-6 in FIG. 1. As indicated by arrow 1, the blood flows into the distal opening 23 and the wall openings 29 of the introducer sheath 13 and flows through the lumen 20 between the outer surface 42 of the balloon catheter 25 and the inner surface 17 of the introducer sheath 13. The openings 29 are advantageous because they can improve flow into the introducer sheath 13, especially when there is little room for blood to flow into only the distal opening 23. From the inside of the introducer sheath 13, the blood flows into the lateral check flow 16 to the stopcock 19 as indicated by arrows 2. The blood cannot flow through the introducer sheath 13 past the lateral check flow 16 because the proximal end 60 is sealed. As indicated by arrows 3, after the blood exits the lateral check flow 16 it passes through the connecting tube 46 to the valve assembly 31. The blood passes through the valve assembly 31 into the balloon catheter 25 as indicated by arrows 4. The blood then travels through the balloon catheter 25 (e.g. through the lumen 41) as indicated by arrows 5 and exits at the tip 40 of the balloon catheter 25 as indicated by arrows 6.

Once the artificial antegrade blood vessel flow is established, a micro catheter 49 may be introduced through the occlusion balloon catheter 25 in a coaxial manner up to the target blood vessel and embolization using standard techniques can be performed. In a high flow blood vessel, the balloon insufflation permits flow control. As there is no retrograde or backflow, there is full control to avoid non-target embolization that may potentially happen at any time during the current embolization techniques using the existing devices available in the market. The balloon 37 may be deflated at any time if needed, and the inherent/native antegrade blood vessel flow can be reestablished immediately.

As a person skilled in the art will readily appreciate, the above description is meant as an illustration of implementation of the principles of this invention. This description is not intended to limit the scope or application of this invention in that the invention is susceptible to modification, variation and change, without departing from the spirit of this invention, as defined in the following claims.

What is claimed is:

1. A reperfusion system comprising:
a sheath having a sheath wall that extends from a proximal portion to a distal portion that has a distal end, the sheath having a lumen that extends to the distal end to define a distal opening of the sheath, the distal portion of the sheath wall having at least one wall opening;
a catheter having a catheter wall that extends from a proximal portion to a distal portion that has a distal end, the catheter having a lumen that extends to the distal end to define a distal opening of the catheter, the catheter being disposed in the lumen of the sheath, the catheter having an expandable member attached to the distal portion; and
a conduit having a first end in fluid communication with the lumen of the sheath and a second end in fluid communication with the lumen of the catheter;
wherein the reperfusion system is configured to allow blood to enter each of the distal opening and the at least one wall opening of the sheath, flow through the sheath, the conduit, and the catheter, and exit through the distal opening of the catheter.

2. The reperfusion system of claim 1 wherein the first end of the conduit is in fluid communication with the lumen of the sheath between the sheath wall and the catheter wall.

3. The reperfusion system of claim 1 further comprising a micro catheter disposed through the opening in the catheter.

4. The reperfusion system of claim 1 further comprising a valve system disposed between the conduit and the catheter.

5. The reperfusion system of claim 1 further comprising a side port integrally formed in the sheath.

6. The reperfusion system of claim 5 further comprising a stopcock on the side port.

7. The reperfusion system of claim 1 further comprising a tip at the distal end of the catheter, the tip having a diameter smaller than a diameter of the catheter.

8. The reperfusion system of claim 1 further comprising an embolic agent carried by the catheter.

9. The reperfusion system of claim 1 wherein the at least one wall opening is a plurality of wall openings.

10. The reperfusion system of claim 9 wherein the plurality of wall openings are circumferentially spaced apart.

11. The reperfusion system of claim 9 wherein the plurality of wall openings are longitudinally spaced apart on the sheath.

12. The reperfusion system of claim 1 wherein the at least one wall opening has a substantially circular or substantially ovular shape.

13. The reperfusion system of claim 1 wherein the at least one wall opening has a substantially polygonal shape.

14. The reperfusion system of claim 1 wherein the expandable member is a balloon.

15. The reperfusion system of claim 14 wherein the catheter includes an inflation lumen that extends to the balloon for inflating the balloon.

16. A reperfusion system comprising:
a sheath having a sheath wall that extends from a proximal portion to a distal portion that has a distal end, the sheath having a lumen that extends to the distal end to define a distal opening of the sheath, the distal portion of the sheath wall having a plurality of wall openings that are one of circumferentially and longitudinally spaced apart;
a balloon catheter having a catheter wall that extends from a proximal portion to a distal portion that has a distal end, the balloon catheter having a lumen that extends to the distal end to define a distal opening of the balloon catheter, the balloon catheter being disposed in the lumen of the sheath, the balloon catheter having an inflatable balloon attached to the distal portion;
a conduit having a first end in fluid communication with the lumen of the sheath between the sheath wall and the catheter wall, and a second end in fluid communication with the lumen of the balloon catheter; and
a micro catheter disposed in the lumen of the balloon catheter;
wherein the reperfusion system is configured to allow blood to enter each of the distal opening and the at least one wall opening of the sheath, flow through the sheath, the conduit, and the balloon catheter, and exit through the distal opening of the balloon catheter.

17. A method of perfusing a blood vessel, the method comprising:
providing a sheath having a sheath wall that extends from a proximal portion to a distal portion that has a distal end, the sheath having a lumen that extends to the distal end to define a distal opening of the sheath, the distal portion of the sheath wall having at least one wall opening;
providing a catheter having a catheter wall that extends from a proximal portion to a distal portion that has a distal end, the catheter having a lumen that extends to the distal end to define a distal opening of the catheter, the catheter having an expandable member attached to the distal portion;
providing a conduit having a first end in fluid communication with the lumen of the sheath and a second end in fluid communication with the lumen of the catheter;
introducing the sheath in body tissue;
advancing the balloon catheter through the sheath to the blood vessel;
expanding the expandable member to prevent blood from flowing in the blood vessel;
allowing the blood to enter each of the distal opening and the at least one wall opening of the sheath, flow through the sheath, the conduit, and the catheter, and exit through the distal opening of the catheter.

18. The method of claim 17 wherein the first end of the conduit is in fluid communication with the lumen of the sheath between the sheath wall and the catheter wall.

19. The method claim 17 wherein the at least one wall opening is a plurality of wall openings.

20. The method of claim 19 wherein the plurality of wall openings are circumferentially spaced apart.

21. The method of claim 19 wherein the plurality of wall openings are longitudinally spaced apart on the sheath.

22. The method of claim 17 wherein the at least one wall opening has a substantially circular or substantially ovular shape.

23. The method of claim 17 wherein the at least one wall opening has a substantially polygonal shape.

24. The method of claim 17 wherein the expandable member is a balloon, and wherein expanding the expandable member comprises inflating the balloon.
